(19) United States
(12) Patent Application Publication
(10) Pub. No.: US 2008/0210242 A1
(43) Pub. Date: Sep. 4, 2008

(54) MASK-NEBULIZER-METERED-DOSE-INHALER ASSEMBLY

(76) Inventors: Marc Alan BURK, Murrieta, CA (US); Gary James Roth, Wake Forest, NC (US); Daniel Patrick Dwyer, Raleigh, NC (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 11/944,972

(22) Filed: Nov. 26, 2007

Related U.S. Application Data
(63) Continuation-in-part of application No. 11/450,457, filed on Jun. 12, 2006.

(51) Int. Cl. A61M 16/06

(52) U.S. Cl. 128/206.21

(57) ABSTRACT

A respiratory therapy mask assembly that includes a mask, a substantially hollow primary extension, and a substantially hollow side extension. The mask assembly further includes a gas inlet adapter configured to direct oxygen into the primary extension when a gas source is connected to the gas inlet adapter. In addition, the mask assembly also includes a valved nebulizer adapter and a metered dose inhaler (MDI) adapter.

### CROSS-REFERENCE TO RELATED APPLICATIONS

This application is related to the U.S. patent application entitled MASK-NEBULIZER ASSEMBLY, filed Jun. 12, 2006, and having a Ser. No. 11/450,457, now pending, the disclosure of which is hereby incorporated by reference in its entirety.

### FIELD OF THE INVENTION

The present invention relates generally to respiratory masks. The present invention also relates generally to methods of manufacturing and using respiratory masks.

### BACKGROUND OF THE INVENTION

Apparatuses that provide a combination of a respiratory mask and a nebulizer are currently available. Each of these apparatuses combine a nebulizer that is secured on an adult or pediatric respiratory mask.

For example, an apparatus that combines a respiratory mask and a nebulizeris illustrated and described in U.S. Pat. No. 5,586,551. Further, U.S. Pat. No. 5,701,886 also discloses a non-rebreather mask assembly for simultaneously delivering oxygen and an aerosolized medication to a patient.

The above notwithstanding, there is a need for novel devices that provide more functionality than provided by the above-discussed apparatuses.

### SUMMARY OF THE INVENTION

The foregoing needs are met, to a great extent, by one or more embodiments of the present invention. For example, the foregoing need is met, at least to some extent, by a respiratory mask assembly according to a first embodiment of the present invention. The respiratory mask assembly includes a mask. The respiratory mask assembly also includes a substantially hollow primary extension including at least a first inlet port, a second inlet port, and a third inlet port, wherein a portion of the primary extension is connected to the mask. In addition, the respiratory mask assembly also includes a substantially hollow secondary extension including a secondary extension inlet port, wherein a portion of the secondary extension is connected to the first inlet port of the primary extension. Further, the respiratory mask assembly also includes a gas inlet adapter connected to the second inlet port of the primary extension, wherein the gas inlet adapter is configured to direct a gas into the primary extension via the second inlet port of the primary extension when a gas source is connected to the gas inlet adapter. Also, the respiratory mask assembly includes a first inhalation substance source adapter connected to the secondary extension inlet port and configured to direct a first inhalation substance from a first inhalation substance source into the primary extension via the secondary extension inlet port when the first inhalation substance is connected to the first inhalation substance adapter, wherein the first inhalation substance adapter includes an outlet portion configured to be secured to the secondary extension inlet port. The respiratory mask assembly also includes a second inhalation substance source adapter connected to the third inlet port of the primary extension, wherein the second inhalation substance source adapter is configured to direct a second inhalation substance from the second inhalation substance source through the third inlet port.

According to another embodiment of the present invention, another respiratory therapy mask assembly is provided. The respiratory therapy mask assembly includes a mask including a mask exhaust port and a mask inlet port and a substantially hollow primary extension including a plurality of inlet ports positioned on an exterior surface thereof, wherein a portion of the substantially hollow primary extension is engaged with the mask inlet port. The respiratory therapy mask assembly also includes a gas inlet adapter connected to a first inlet port of the primary extension, wherein the gas inlet adapter is configured to direct oxygen into the primary extension via the first inlet port of the primary extension when a gas source is connected to the gas inlet adapter. In addition, the respiratory therapy mask assembly also includes a metered dose inhaler adapter connected to a second inlet port of the primary extension and configured to direct an inhalation substance from a metered dose inhaler connected to the metered dose inhaler adapter into the primary extension when the metered dose inhaler is connected to the metered dose inhaler adapter.

There has thus been outlined, ratherbroadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 illustrates a mask assembly according to one embodiment of the present invention, wherein the assembly includes an elongated hollow pipe for simultaneously delivering an inhalation substance (e.g., an aerosol) and oxygen to a mask, with portions of the pipe and mask partially cut away to show component connections;

FIG. 2 illustrates a therapy mask assembly according to another embodiment of the present invention, wherein the assembly includes a therapy mask, a nebulizer, and a gas reservoir bag;

FIG. 3 illustrates a sectional view of the elongated hollow pipe illustrated in FIG. 2 showing a gas inlet pipe adapter and a valved nebulizer adapter positioned in the hollow pipe; and

FIG. 4 illustrates a respiratory therapy mask assembly according yet another embodiment of the present invention.

FIG. 5 illustrates a sectional view of a portion of the respiratory therapy mask assembly illustrated in FIG. 4.

FIG. 6 is a perspective view of the mask assembly illustrated in FIG. 4 when an a Metered Dose Inhaler (MDI) and no nebulizer is connected thereto.

FIG. 7 is a perspective view of the mask assembly illustrated in FIG. 4 when a nebulizer and no MDI is connected thereto.

### DETAILED DESCRIPTION

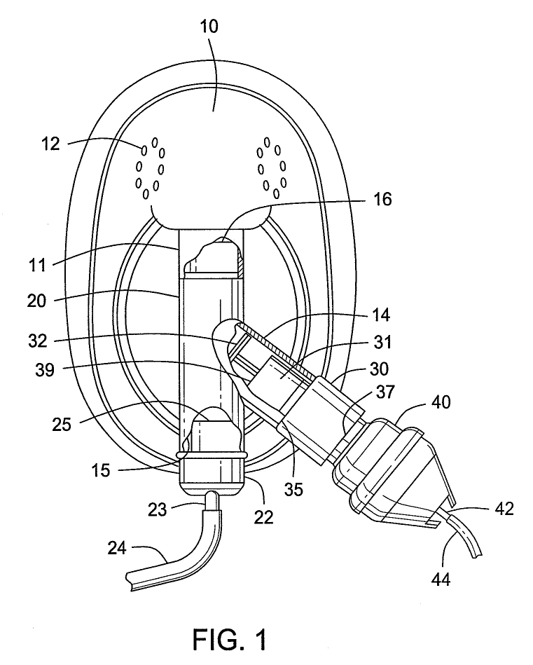

The invention will now be described with reference to the drawing figures, in which like reference numerals refer to like parts throughout. One embodiment of the present invention is illustrated in FIG. 1, which illustrates a mask assembly according to one embodiment of the present invention, wherein the assembly includes an elongated hollow pipe 20 for simultaneously delivering an inhalation substance and oxygen to a mask 10, with portions of the pipe 20 and mask 10 partially cut away to show component connections. According to various embodiments of the present invention, the inhalation substance may take the form of one or more of the following: an aerosol, a vapor, a mist, a powder, a medicament, and particles (including nanoparticles).

As illustrated in FIG. 1 the respiratory therapy mask assembly includes the face mask 10, the elongated hollow pipe 20 secured on the mask via a gas port 11, a nebulizer adapter assembly 30, and a nebulizer 40. The mask 10 is provided with exhalation ports 12 of varying configurations on each side of the mask, near a patient's nose and mouth. The exhalation ports 12 allow patient breath to be vented to the atmosphere during expiration, and also allow some room air to be drawn into the mask during inspiration, depending on peak inspiratory flow.

The mask design illustrated in FIG. 1 is often referred to as a medium concentration mask. The mask 10 illustrated in FIG. 1 is elongated to fit under the chin of a patient. However, a shorter mask (i.e., a mask that does not fit under the chin of the patient), or a mask with a geometry other than what is illustrated in FIG. 1 may also be used according to certain embodiments of the present invention.

An inlet port 11 on the mask is configured to receive an upper end 16 of the elongated hollow pipe 20, as is visible through the cutaway portion of the inlet port 11 illustrated in FIG. 1. At the lower end of elongated hollow pipe 20 is secured a gas inlet pipe adapter 22. The upper end 25 of the gas inlet pipe adapter 22 is shown through the cutaway of the lower end of elongated hollow pipe 20.

The gas inlet pipe adapter 22 is provided with an integral gas inlet pipe 23, to which is secured a gas tubing 24. According to certain embodiments of the present invention, the gas tubing 24 directs oxygen or an oxygen-containing respiratory gas from a source (not illustrated in FIG. 1) to the respiratory therapy mask 10.

In the embodiment of the present invention illustrated in FIG. 1, the gas inlet pipe adapter 22 is provided with a collar 15. In operation, the collar 15 limits the extent of possible insertion of the pipe adapter 22 into the elongated hollow pipe 20. However, according to other embodiments of the present invention, the collar 15 is not included in the mask assembly.

The installed pipe adapter 22 illustrated in FIG. 1 is in movable engagement to the end of the elongated hollow pipe 20. Thus, although the second pipe adapter 22 is in gas sealed fit with the lower end of the pipe 20 (e.g., in force fit engagement), the adapter 22 is also rotatably secured so that the pipe adapter 22 and integrated gas inlet pipe 23 can be conveniently rotated in a desired direction to avoid interference with an attached angled side pipe extension 14, the nebulizer adapter assembly 30, and/or the attached nebulizer 40 illustrated in FIG. 1. It is thus possible to position the gas delivery tubing 24 at desired angles without interfering with other equipment, tubing, and/or components.

The angled side pipe extension 14 illustrated in FIG. 1 is also substantially hollow and provides a second inlet for directing fluid through the hollow pipe 20 to the respiratory mask 10. The inlet port 11 and angled side pipe extension 14 are configured to receive the valved nebulizer adapter assembly 30. As illustrated in FIG. 1, an inhalation substance pipe 31 included in the valved nebulizer adapter assembly 30 extends into the angled pipe extension 14 to a position which allows an inhalation substance generated by the nebulizer 40 to be introduced into the elongated hollow pipe 20.

According to certain embodiments of the present invention, the valved nebulizer adapter assembly 30 is provided with either a shoulder 35 or other equivalent feature or component. The shoulder 35 is then used to limit the insertion of the adapter assembly 30 to a position which effectively secures the adapter assembly 30 in the angled side pipe extension 14. The mating or engagement of the nebulizer adapter assembly 30 and the side pipe 14 may include, for example, a force fit, a threaded engagement, or any other form of engagement or mating that will become apparent to one of skill in the art upon practicing one or more embodiments of the present invention. For example, according to certain embodiments of the present invention, the nebulizer adapter assembly 30 is provided with a guide member or protuberance (not illustrated) that is configured to be engaged in a slot or recess (not illustrated) formed at the inlet port of the angled side pipe 14.

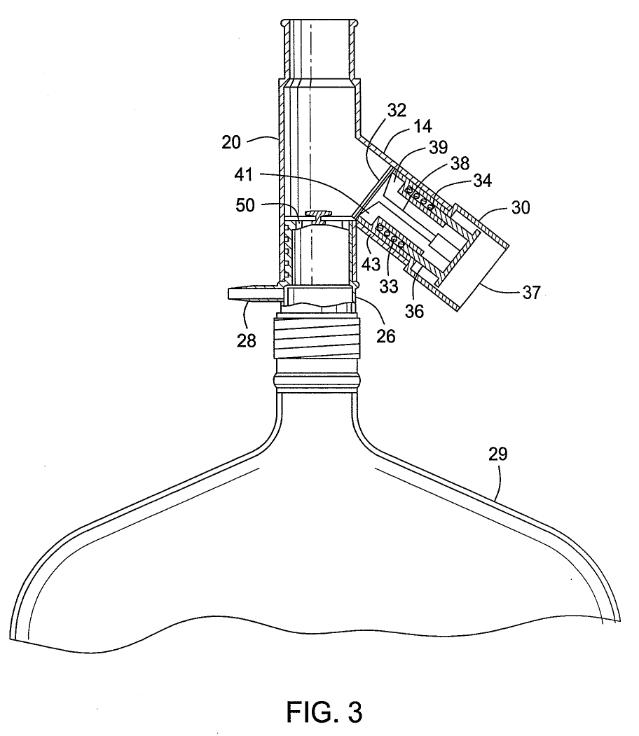

With reference to FIG. 3, the above-discussed valved nebulizer adapter assembly 30 includes an inlet port 37 that is configured to receive an outlet pipe (not illustrated) and port (not illustrated) of a nebulizer. The assembly 30 also includes an inhalation substance outlet port 39.

According to certain embodiments of the present invention, the valved nebulizer adapter assembly 30 also includes a valve 32 for closing the inhalation substance outlet (i.e., exhaust) port 39. As illustrated in FIG. 3, the valve 32 is biased to a closed position by a spring 34 that is housed in a chamber located between an inside wall 41 and an outer wall 43 of the assembly 30. In operation, the typically closed valve 32 is opened by forcing the outlet pipe (not illustrated) of a nebulizer against a valve actuator 36 which compresses the spring 34 and urges valve 32 to an open position via a valve stem 38 that is structurally connected to a valve actuator 36.

A more complete description of a valve nebulizer adapter assembly that may be used according to certain embodiments of the present invention is disclosed in U.S. Pat. No. 6,725,858, which is hereby incorporated herein by reference in its entirety. Of course, other valved nebulizer adapter assembly designs may also be used. For example, the assembly described in U.S. Pat. No. 4,951,661, which is also hereby incorporated herein by reference in its entirety, may be used.

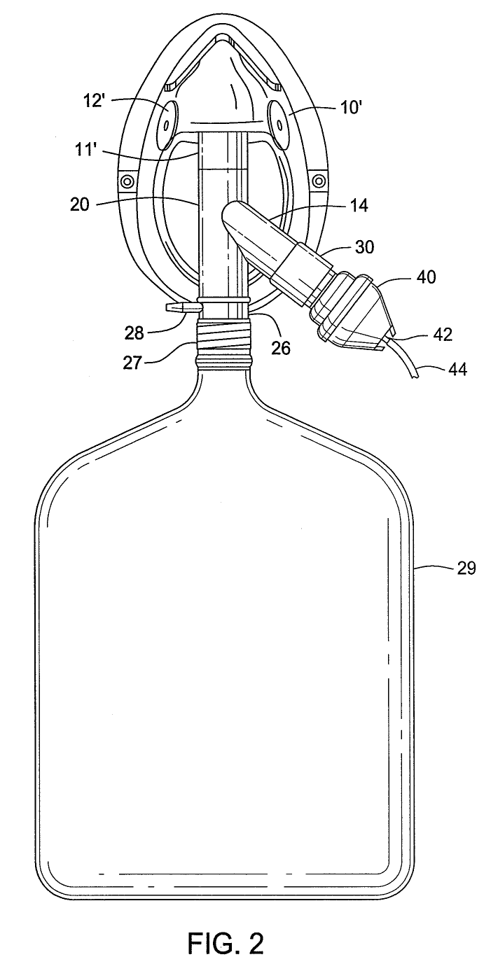

According to certain embodiments of the present invention, the nebulizer 40 is inserted into the inhalation substance inlet port 37 as illustrated in FIGS. 1 and 2. As also illustrated in FIGS. 1 and 2, the nebulizer 40 is provided with a gas supply pipe 42 and tubing 44. Upon practicing one or more embodiments of the present invention, one of skill in the art will recognize that many nebulizers suitable for delivering an inhalation substance for patient respiratory therapy or other medical treatment may be used as the above-discussed nebulizer 40. For example, the nebulizers described in U.S. Pat. Nos. 4,588,129 and Re. 33,642, which are hereby incorporated herein by reference in their entirety, may be used as the nebulizer 40.

FIG. 2 illustrates a therapy mask assembly according to another embodiment of the present invention, wherein the assembly includes a therapy mask 10′, a nebulizer 40, and a gas reservoir bag 29. As illustrated in FIG. 2, the reservoir bag 29 is secured adjacent to the lower end of the elongated hollow pipe 20.

FIG. 3 illustrates a sectional view of the elongated hollow pipe 20 illustrated in FIG. 2 and also shows a gas inlet pipe adapter 26 and a valved nebulizer adapter positioned in the hollow pipe. The assembly illustrated in FIG. 3 also includes a one-way valve 50 that, if it were illustrated in FIG. 2, would be positioned between the gas reservoir bag 29 and the mask 10′.

FIG. 3 also illustrates that, according to certain embodiments of the present invention, the adapter 26 includes an integrally formed gas inlet pipe 28 that extends outwardly along the length of the adapter 26. The upper end of the adapter 26 in FIG. 3 is secured into the lower end of the elongated hollow pipe 20 and the lower end of the adapter 26 is secured to the reservoir bag 29. The upper end of adapter 26 illustrated in FIG. 3 is also provided with one-way valve 50 in the form of a flexible disc or wafer which allows a gas to be directed at least substantially only one way into the elongated hollow pipe 20 from the gas inlet pipe 28 and the reservoir bag 29.

The mask assembly design discussed above and illustrated in FIGS. 2 and 3 is referred to as a non-rebreathing mask. Alternatively, the one-way valve 50 may be eliminated, in which case the mask assembly operates as a partial rebreathing mask or a high concentration mask. Either type of mask design is within the scope of certain embodiments of the present invention.

In the embodiment of present invention illustrated in FIG. 2, a pair of one-way valves 12′ are secured on the mask 10′ to prevent entrainment of atmospheric air during patient inspiration. Also, the pair of one-way valves 12′ ultimately provides for a higher oxygen concentration to be delivered to the patient, as compared to a mask in which one or more of the one-way mask valves 12′ are omitted. As also is shown in FIG. 2, the reservoir bag 29 is secured to the lower end of the adapter 26 with tape 27.

The respiratory therapy mask assemblies described herein, regardless of the specific assembly configuration or embodiment, typically allow for simultaneous respiratory gas and inhalation substance delivery to a patient. These assemblies also allow for selective removal of a nebulizer from the assembly without interrupting the respiratory gas flow and still maintaining the integrity of the gas delivery system by closure of the inhalation substance piping by simply detaching and removing the nebulizer. Such a feature does not require the user, patient, or therapist to close the inhalation substance delivery limb of the assembly. Moreover, there is no additional requirement for introducing the inhalation substance other than attaching the nebulizer. As will be appreciated by one of skill in the art upon practicing one or more embodiments of the present invention, this substantially simplifies operation and convenience of the apparatus.

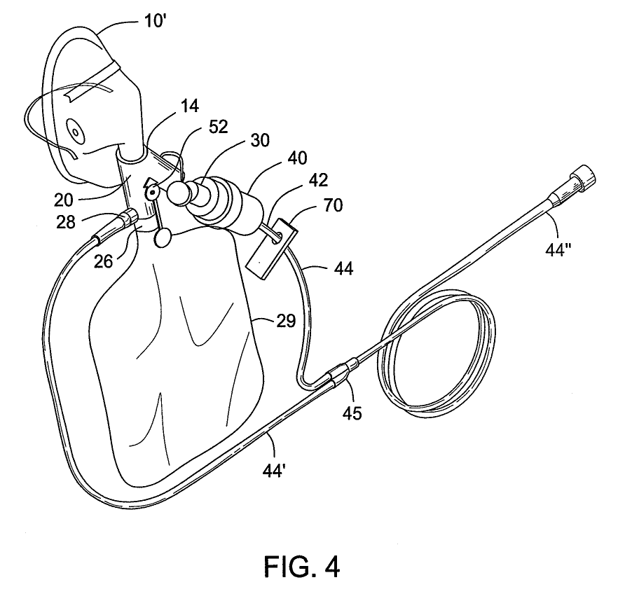

FIG. 4 illustrates a respiratory therapy mask assembly according yet another embodiment of the present invention. Like the assembly illustrated in FIGS. 2 and 3, the assembly illustrated in FIG. 4 includes a mask 10′ that includes one-way valves 12′ that prevent entrainment of atmospheric air during patient inspiration. The mask 10′ also has an elongated hollow pipe 20 connected thereto and protruding therefrom. Although the pipe 20 is illustrated as substantially cylindrical in FIG. 4, substantially or at least partially hollow extensions of other cross-sectional geometries may also be used in alternate embodiments of the present invention. It should also be noted that, as used herein, the term “extension” includes, for example, a pipe, hose, channel, duct, line, tube, or other similar conduit.

The pipe 20 illustrated in FIG. 4 has an angled side pipe extension 14 protruding therefrom. The side pipe extension 14 is connected to the hollow pipe 20 on a proximate end thereof and interfaces with a nebulizer adapter assembly 30 on a distal end thereof. In turn, the nebulizer adapter assembly is connected to a nebulizer 40.

Connected to a side of the nebulizer 40 not connected to the nebulizer adapter assembly 30 is a gas supply pipe 42 and tubing 44. In operation, gas (e.g., oxygen) flows from a gas source (not illustrated but connected to the tubing 44) and through the tubing and gas supply pipe 42. Then, as the gas continues to flow through the nebulizer 40, nebulizer adapter assembly 30 and into the mask 10′, the gas assists in delivering medication (typically as an inhalation substance) from the nebulizer 40 to a patient wearing the mask 10′.

As illustrated in FIG. 4, according to certain embodiments of the present invention, the tubing 44 is connected to a splitter 45. The splitter 45 accommodates not only the above-discussed length of tubing 44 that is connected to the nebulizer 40 but also a second length of tubing 44′ that will be discussed below and a third length of tubing 44″ (i.e., a gas supply line) that is connected to the gas source (not illustrated). In operation, the same gas source can be used to supply gas to all three lengths of tubing 44, 44′, 44″. However, according to other embodiments of the present invention, the splitter 45 may be removed and/or different gas sources may be connected to each of the first and second lengths of tubing 44, 44′.

Returning to the top portion of FIG. 4, connected to the end of the hollow pipe 20 that is opposite the mask 10′ is the proximate end of an adapter 26. The distal end of the adapter 26, in turn, is connected to a reservoir bag 29.

A gas inlet pipe 28 is connected to the adapter 26. The gas inlet pipe 28, in turn, is connected to the second length of tubing 44′ and a gas supply pipe 44″. In operation, a gas (e.g., oxygen) flows through the gas supply pipe 44″, the second length of tubing 44′, and either through the hollow pipe 20 or into the reservoir bag 29.

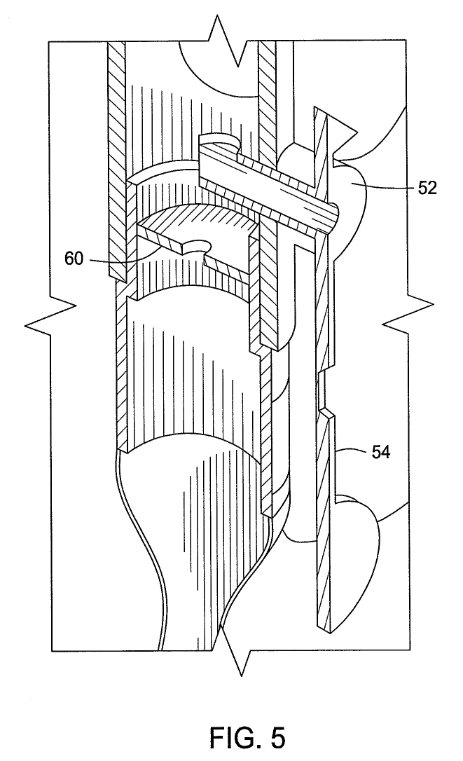

Also located between the proximate and distal ends of the adapter 26 is a metered dose inhaler (MDI) adapter or port 52. FIG. 5 illustrates a cross-sectional view of the portion of the hollow pipe 20 adjacent to the MDI port 52. As illustrated in FIGS. 4 and 5, surrounding the MDI port 52 is a port cover 54.

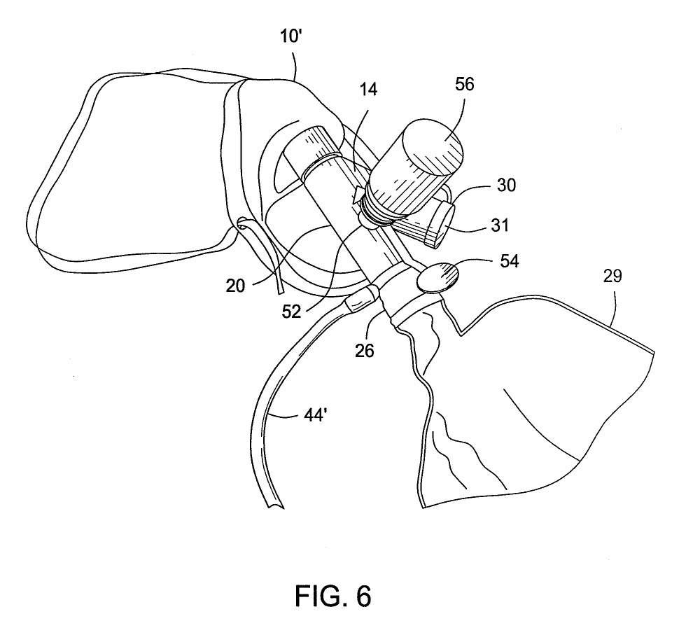

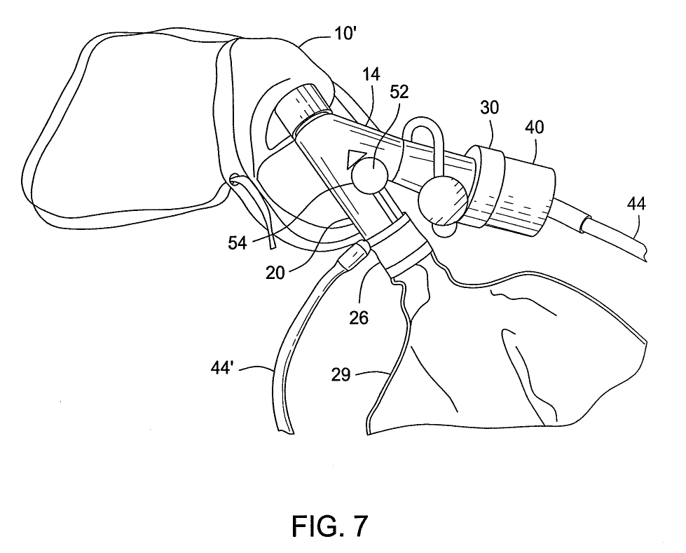

FIG. 6 is a perspective view of the mask assembly illustrated in FIG. 4 when an MDI 56 and no nebulizer is connected thereto. FIG. 7 is a perspective view of the mask assembly illustrated in FIG. 4 when a nebulizer 40 and no MDI is connected thereto. As illustrated in FIG. 7, the port cover 54 is folded upon itself and seals the port 52 such that no gas flows therethrough. In contrast, in FIG. 6, the nebulizer adapter assembly 30 is sealed by a cap 31 and the port cover 54 is unfolded and the MDI 56 is connected to the hollow pipe 20.

According to embodiments of the present invention, either a commercially available or specially designed MDI canister may be used in conjunction with the mask assembly illustrated in FIG. 4. In FIG. 6, once the MDI has been actuated, a metered dose of a medicament enters the hollow pipe 20 through the port 52. Then, the gas flowing through the second length of tubing 44′ delivers the medicament to the patient wearing the mask 10′.

As illustrated in FIG. 5, inside of the hollow pipe 20, below the port 52, and above the gas inlet pipe 28 is a one-way valve 60. The one-way valve 60 prevents flow of gas from the side pipe extension 14 to the reservoir bag 29 but allows flow of gas from second length of tubing 44′ to the mask 10′.

According to certain embodiments of the present invention, either a single gas supply line or dual gas supply lines may be used. FIG. 4 illustrates one embodiment of the present invention wherein the tubing 44 can function as a single gas supply line and wherein the splitter 45 may act as a specially modified forked connector.

According to some of these embodiments, the splitter 45 includes a reduced orifice for oxygen supply to the mask 10 and a larger orifice supplying gas to the nebulizer 40. The reduced orifice size for the mask gas supply line 44″ powers the nebulizer with adequate flow when both devices are connected to a single gas source.

The single supply line setup discussed above is typically used in applications where there is only one oxygen port, such as in an Emergency Medical Treatment (EMT) vehicle, and compressed air or a second oxygen source is unavailable to drive the nebulizer 40. The single drive line configuration also typically incorporates a gas flow valve 70 which stops flow to the nebulizer 40 when not in use. However, according to other embodiments of the present invention, dual gas supply lines are provided, one for the nebulizer 40 and one for the mask 10 oxygen supply, both of which are controlled independently.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents maybe resorted to, falling within the scope of the invention.

What is claimed is:

1. A respiratory therapy mask assembly, comprising:
a mask;
a substantially hollow primary extension including at least a first inlet port, a second inlet port, and a third inlet port, wherein a portion of the primary extension is connected to the mask;
a substantially hollow secondary extension including a secondary extension inlet port, wherein a portion of the secondary extension is connected to the first inlet port of the primary extension;
a gas inlet adapter connected to the second inlet port of the primary extension, wherein the gas inlet adapter is configured to direct a gas into the primary extension via the second inlet port of the primary extension when a gas source is connected to the gas inlet adapter;
a first inhalation substance source adapter connected to the secondary extension inlet port and configured to direct a first inhalation substance from a first inhalation substance source into the primary extension via the secondary extension inlet port when the first inhalation substance is connected to the first inhalation substance adapter, wherein the first inhalation substance adapter includes an outlet portion configured to be secured to the secondary extension inlet port; and
a second inhalation substance source adapter connected to the third inlet port of the primary extension, wherein the second inhalation substance source adapter is configured to direct a second inhalation substance from the second inhalation substance source through the third inlet port.

2. The respiratory therapy mask assembly of claim 1, further comprising:
a valved nebulizer adapter assembly connected to the side extension port and configured to direct an inhalation substance from a nebulizer into the primary extension via the side extension inlet port when the nebulizer is connected to the valved nebulizer adapter assembly.

3. The respiratory therapy mask assembly of claim 2, wherein the valved nebulizer assembly adapter assembly comprises:
an inhalation substance outlet portion configured to be secured to the side extension inlet port; and
a valve biased to close the inhalation substance outlet portion and configured to open upon insertion of the nebulizer.

4. The respiratory therapy mask assembly of claim 2, wherein the valved nebulizer assembly adapter assembly comprises:
a third inlet port in the primary extension configured to direct a second inhalation substance from a source connected to the third inlet port.

5. The respiratory therapy mask of claim 4, wherein a metered dose inhaler adapter is connected to the third inlet port and wherein the metered dose inhaler adapter comprises a port configured to accommodate flow of an inhalation substance medicament from a metered dose inhaler to the primary extension when the metered dose inhaler is connected to the metered dose inhaler adapter and actuated.

6. The respiratory therapy mask of claim 5, further comprising:
a cover configured to seal the port when the metered dose inhaler is not connected to the metered dose inhaler adapter.

7. The respiratory therapy mask of claim 2, further comprising:
a seal configured to seal the valved nebulizer adapter assembly when the nebulizer is not connected to the valved nebulizer adapter assembly.

8. The respiratory therapy mask of claim 7, wherein the seal comprises a cap screwable unto a portion of the valved nebulizer adapter assembly.

9. The respiratory therapy mask of claim 2, further comprising:
a first length of tubing connected to the gas inlet adapter;
a second length of tubing connected to the valved nebulizer adapter assembly.

10. The respiratory therapy mask of claim 9, further comprising;
a splitter connected to the first length of tubing and the second length of tubing, wherein the tubing is configured to allow the gas from the gas source to flow through both the first length of tubing and the second length of tubing.

11. The respiratory therapy mask of claim 9, wherein the splitter comprises:
a first orifice and a second orifice, wherein the first orifice and the second orifice are of different sizes.

12. The respiratory therapy mask assembly of claim 1, further comprising:
a gas reservoir connected to the gas inlet adapter.

13. The respiratory therapy mask assembly of claim 1, further comprising:
a one-way valve connected to the gas inlet adapter.

14. The respiratory therapy mask assembly of claim 1, wherein the mask includes a the mask exhaust port and wherein the mask exhaust port includes a valve.

15. A respiratory therapy mask assembly, comprising:
a mask including a mask exhaust port and a mask inlet port;
a substantially hollow primary extension including a plurality of inlet ports positioned on an exterior surface thereof, wherein a portion of the substantially hollow primary extension is engaged with the mask inlet port;
a gas inlet adapter connected to a first inlet port of the primary extension, wherein the gas inlet adapter is configured to direct oxygen into the primary extension via the first inlet port of the primary extension when a gas source is connected to the gas inlet adapter;
a metered dose inhaler adapter connected to a second inlet port of the primary extension and configured to direct an inhalation substance from a metered dose inhaler connected to the metered dose inhaler adapter into the primary extension when the metered dose inhaler is connected to the metered dose inhaler adapter.

16. The respiratory therapy mask of claim 15, wherein the metered dose inhaler adapter comprises a port configured to accommodate flow of a medicament from the metered dose inhaler to the primary extension when the metered dose inhaler is connected to the metered dose inhaler adapter and actuated.

17. The respiratory therapy mask of claim 16, further comprising:
a cover configured to seal the port when the metered dose inhaler is not connected to the metered dose inhaler adapter.

18. The respiratory therapy mask assembly of claim 16, further comprising:
a gas reservoir connected to the gas inlet adapter.

19. The respiratory therapy mask assembly of claim 16, further comprising:
a one-way valve connected to the gas inlet adapter.

20. The respiratory therapy mask assembly of claim 15, wherein the mask exhaust port includes a valve.
